(19) United States
(12) Patent Application Publication
(10) Pub. No.: US 2010/0174099 A1
(43) Pub. Date: Jul. 8, 2010

(54) Propylene oxide reactor gas distribution system

(75) Inventors: Arsam Behkish, Broomall, PA (US); Rafael Espinoza, Tulsa, OK (US); John H. Speidel, JR., Media, PA (US)

Correspondence Address: BASELL USA INC., NEWTOWN SQUARE CENTER, 3801 WEST CHESTER PIKE, BLDG. B, NEWTOWN SQUARE, PA, 19073, US

(73) Assignee: Lyondell Chemical Technology, L.P., Greenville, DE (US)

(21) Appl. No.: 12/319,247

(22) Filed: Jan. 5, 2009

(51) Int. Cl. C07D 301/02; B01J 19/00

(52) U.S. Cl. 549/518; 422/129

(57) ABSTRACT

A device for reacting fluids comprising:
a reactor;
a first inlet for transporting a first fluid into the reactor;
a first tube system contained within the reactor and connected to and communicating with the first inlet for receiving the first fluid from the first inlet, the first tube system comprising at least one first tube in the form of a ring,
   the at least one first tube comprising outlets for releasing the first fluid into the reactor;
a second inlet for transporting a second fluid into the reactor; and
a second tube system contained within the reactor and connected to and communicating with the second inlet for receiving the second fluid from the second inlet, the second tube system comprising at least one second tube in the form of a ring,
   the at least one second tube comprising outlets for releasing the second fluid into the reactor;
      wherein at least one first tube and at least one second tube are concentric, and the outlets for releasing the first fluid from the at least one first tube and the outlets for releasing the second fluid from the at least one second tube are positioned so that, upon release of the first and second fluids, the first and second fluids are directed towards each other.

### FIELD OF THE INVENTION

The present subject matter relates generally to a gas distribution system for mixing reactants in a slurry bubble column reactor. More particularly, the present subject matter relates to a gas distributor and a process for its use to produce propylene oxide in a slurry bubble column reactor.

### BACKGROUND OF THE INVENTION

Propylene oxide has long been produced commercially via the chlorohydrin or epoxide processes. In the chlorohydrin process, propylene and chlorine are reacted in the presence of water to form propylene chlorohydrins. Propylene oxide is recovered by subsequently reacting the propylene chlorohydrins with sodium or calcium hydroxide. The chlorohydrins process is limited in that the production of propylene oxide is accompanied by the undesirable production of various salts, and the need to separate propylene oxide from large quantities of water. Epoxidation reactions have also been used, where ethylbenzene is reacted with oxygen to produce ethylbenzene hydroperoxide, which is then reacted with propylene to form propylene oxide. A phenylmethylcarbinol co-product is then dehydrated to form styrene. Alternately, isobutene can be used to form a tert-butyl alcohol co-product which can be converted to methyl tertiary butyl ether (MTBE). However, these processes are limited by the market demand for the various co-products that are formed. As a result, other processes have been proposed to overcome the above limitations by the direct oxidation of propylene with oxygen and hydrogen in a solvent, in the presence of a catalyst (U.S. Pat. Nos. 7,138,535, 7,238,817, 7,279,145, and 5,973,171). U.S. patent application Ser. No. 12/079,823 describes a process for producing propylene oxide by reacting propylene, oxygen, and hydrogen in a slurry comprising a catalyst and a solvent, the disclosure of which is hereby incorporated by reference.

Production of propylene oxide in the so-called direct propylene oxide process requires routing oxygen and propylene/hydrogen reactant streams to the reactor separately, to avoid the presence of flammable mixtures that would occur upon mixing. Introducing gaseous reactants into a slurry bubble column reactor (SBCR) in such a way as to ensure reliability and efficiency is challenging, since the reaction involves distribution of the gaseous reactants into a slurry of a liquid solvent containing solid catalyst particles.

Various gas distribution systems have been described in the literature. U.S. Pat. Nos. 5,620,670 and 5,621,155 describe distributors used in Fischer-Tropsch reactors consisting of orifices or porous metal spargers on concentric rings or torroidal manifolds. International Publication Number WO2005/094979 discloses a slurry bubble column reactor gas distributor in the shape of concentric rings or tubular toroids conforming to the bottom of the reactor. Nozzles on the distributor preferably have an inclination of 45° or less to the vertical, with the openings of the nozzles directed perpendicular towards the surface of the reactor bottom. U.S. Pat. Nos. 4,785,123 and 4,883,889 disclose processes for producing alkylene oxides by reacting alkanes, alkylenes or mixtures thereof with an oxygen-containing gas in the presence of a molten nitrate salt using a co-axially mounted feed gas tubes and a sparger. However, a continuing need exists for gas distribution equipment that efficiently and reliably distributes gaseous reactants for the production of propylene oxide.

Accordingly, it is an object of the present subject matter to provide improved gas distribution in a process for the production of propylene oxide using a gas distributor containing concentric rings, where an oxygen-containing reactant and a propylene/hydrogen-containing reactant are introduced into a reactor through outlets in separate rings, where the outlets in the separate rings are directed towards each other.

### SUMMARY OF THE INVENTION

The above and other objects, which will become apparent to one skilled in the art upon a reading of this disclosure, are attained by the present subject matter, one aspect of which is:

A device for reacting fluids comprising:

a reactor;

a first inlet for transporting a first fluid into the reactor;

a first tube system contained within the reactor and connected to and communicating with the first inlet for receiving the first fluid from the first inlet, the first tube system comprising at least one first tube in the form of a ring,
the at least one first tube comprising outlets for releasing the first fluid into the reactor;

a second inlet for transporting a second fluid into the reactor; and

a second tube system contained within the reactor and connected to and communicating with the second inlet for receiving the second fluid from the second inlet, the second tube system comprising at least one second tube in the form of a ring,
the at least one second tube comprising outlets for releasing the second fluid into the reactor,
   wherein at least one of the first tubes and at least one of the second tubes are concentric, and the outlets for releasing the first fluid from the at least one first tube and the outlets for releasing the second fluid from the at least one second tube are positioned so that, upon release of the first and second fluids, the first and second fluids are directed towards each other.

Another aspect of the present subject matter is:

A process comprising:

feeding a first fluid into a reactor containing a solvent and a catalyst, through a first tube system contained within the reactor, the first fluid comprising oxygen; and

feeding a second fluid into the reactor through a second tube system contained within the reactor, thereby forming propylene oxide, the second fluid comprising propylene and hydrogen,

wherein the first tube system and second tube system are positioned such that, upon release into the reactor, the first and second fluids are directed towards each other.

### BRIEF DESCRIPTION OF THE DRAWINGS

FIG. 1 is a vertical cross section of the reactor, reactor inlets and gas distributor containing a single pair of tubes that are in the same horizontal plane.

FIG. 1A is an enlarged fragmentary view of the first and second tubes, and outlets on the first and second tubes.

FIG. 2 is a cross section view taken along 2-2 of the first and second tubes and outlets on the first and second tubes.

FIG. 3 is a top plan view of a gas distributor containing 3 ring pairs.

FIG. 4 is a top perspective view of a gas distributor containing 3 ring pairs.

FIGS. 5A-5C are vertical cross sections through a reactor having a 3 ring pair gas distributor.

FIG. 6 is a shroud covering a tube outlet in the shape of an orifice.

FIG. 7 is a schematic of control valves located in the first and second tube systems.

FIG. 8 illustrates an analyzer installed in the sidewall of the reactor.

FIG. 9 is a vertical cross section of the reactor, reactor inlets and gas distributor containing a single pair of tubes that are not in the same horizontal plane.

FIG. 9A is an enlarged fragmentary view of the first and second tubes, and outlets on the first and second tubes.

FIG. 10 is a cross section view taken along 2-2 of the first and second tubes and outlets on the first and second tubes.

### DETAILED DESCRIPTION OF THE INVENTION

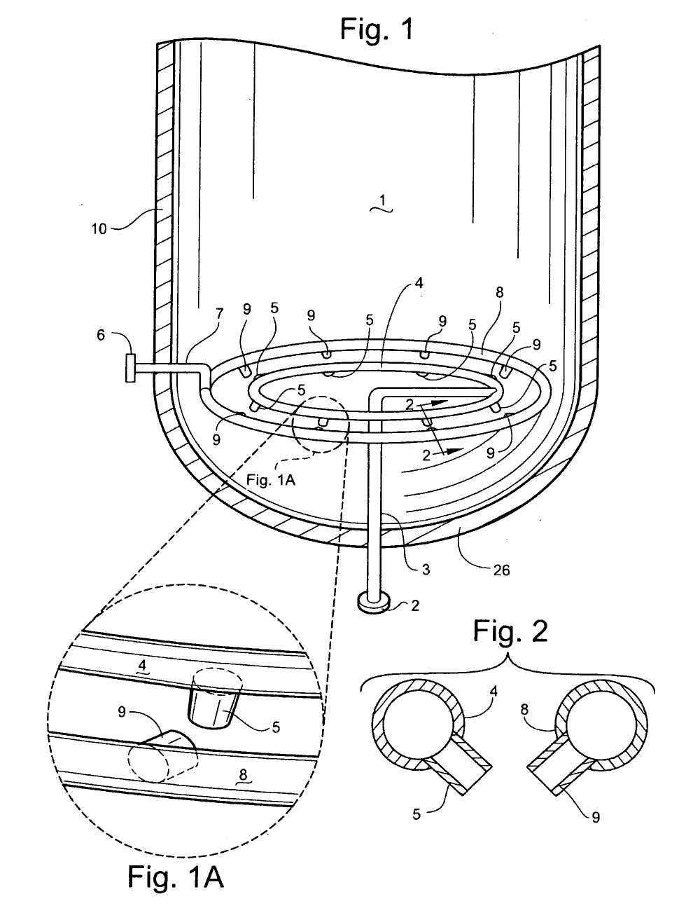

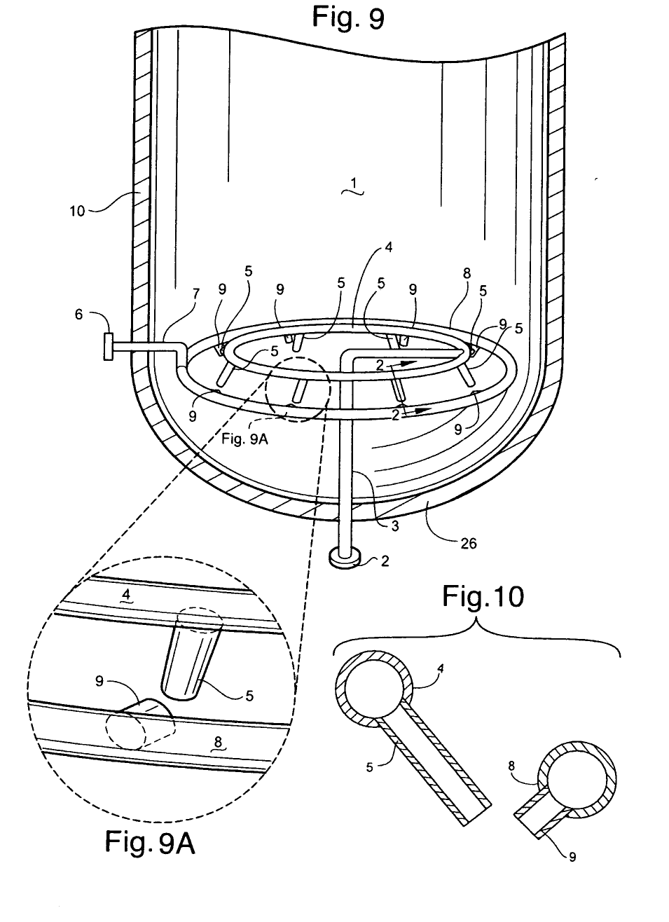

The present subject matter will be described in detail with reference to the drawings. Referring to FIGS. 1 and 9, a slurry bubble column reactor 1 contains a slurry of solvent, catalyst and buffer. Reactor 1 is preferably cylindrical in shape and preferably has a height-to-diameter ratio of greater than 3:1. The head design of the reactor 1 can be any type conventionally used, consistent with the reactor geometry and operating conditions. The materials of construction for the reactor 1 can be any that are compatible with the feed and product streams processed within the reactor at the operating pressures and temperatures. Preferably, the reactor is a column having a top portion, a bottom portion, and a sidewall portion, with the bottom portion having a reactor bottom head 26. For the purposes of this specification, the term “top portion” means the portion of the reactor above the midpoint of the reactor. The term “bottom portion” means the portion of the reactor below the midpoint of the reactor.

The reactor 1 preferably operates at a pressure of from 100 to 800 psig, more preferably 100 to 500 psig, at temperatures of from 30 to 100° C., more preferably 40 to 60° C. The reactor 1 can also be equipped with a variety of conventional internal auxiliary equipment such as baffles, filters and heat transfer tubes.

The solvent preferably includes alcohols, aromatic and aliphatic solvents such as toluene and hexane, nitrites such as acetonitrile, ethers esters, ketones, water and mixtures thereof. More preferably, solvents include methanol, water and mixtures thereof. The catalyst preferably contains a transition metal zeolite and a noble metal, and is preferably present in the form of particles having a mean mass diameter of from 10 to 500 μm, more preferably, from 20 to 100 μm. Preferably, the catalyst is present in the slurry at a concentration of between 1 and 40 wt %, more preferably, between 5 and 20 wt %.

The buffer preferably includes salts of oxyacids, more preferably alkali metal phosphates, ammonium phosphates and ammonium hydroxide, and are present in the solvent at concentrations from 0.0001 M to 1 M.

An oxygen-containing first feed stream and a second feed stream containing propylene, hydrogen, ballast gas and inert gases, are separately introduced into reactor 1 through a first inlet 6 and a second inlet 2. The first and second feed streams can be introduced into reactor 1 through either inlet. However, the feed points are separate to avoid flammability problems associated with contact of the feed streams prior to their introduction into the slurry-filled reactor 1. Oxygen in the first feed stream is present in a concentration of greater than 10 mol %. Preferably, it is present in an amount greater than 90 mol %. The remainder of the first feed stream includes inert gases such as nitrogen, helium, argon, carbon dioxide and mixtures thereof. The second feed stream can include blends of fresh hydrogen and propylene, recycled gases from downstream separation equipment, ballast gas, and oxygen at levels less than 10 mol %, preferably, less than 7 mol %. Recycled gases are primarily hydrogen, propylene, and ballast gas. Ballast gas is added to maintain a vapor space in reactor 1, and can include saturated hydrocarbons with 1-4 carbon atoms, e.g., methane, ethane, propane, and n-butane. Hydrogen is present in the second feed stream in such an amount that the molar ratio of hydrogen to oxygen in the total of the feed streams is preferably 0.01 to 10.0, more preferably 0.2 to 2.0. Propylene is present in the second feed stream in such an amount that the molar ratio of oxygen to propylene in the total of the feed streams is 0.05 to 1.0, preferably 0.1 to 0.67. The molar ratio of propylene to inert gas is preferably between 0.05 to 100.0, more preferably between 0.05 to 20.0.

Preferably, the superficial gas velocity in reactor 1 is in the range of 0.05 to 0.60 m/s, more preferably, in the range of 0.08 to 0.2 m/s. Reactor 1 operates in a heterogenous flow regime (churn turbulent flow), where large bubbles or agglomerates of bubbles form and travel upward at high velocity, mainly in the axis of the vessel.

The first feed stream preferably flows through the first inlet 6, which is preferably located in reactor sidewall 10. The first inlet 6 is connected to and communicates with the first tube system 7. First tube system 7 includes tubing that routes the first feed stream from the first inlet 6 to the first tube 8, which is in the form of a ring. The portion of the first tube system 7 that routes the first feed stream to the first tube 8 can be configured in any manner consistent with the location and orientation of the first inlet 6, first tube 8, second tube 4 and second tube system 3, and can include multiple tubes, tube manifolds or combinations thereof.

The second feed stream preferably flows through the second inlet 2, which is preferably located in the reactor bottom head 26. The second inlet 2 is connected to and communicates with the second tube system 3. Second tube system 3 includes tubing that routes the second feed stream from the second inlet 2 to the second tube 4, which is in the form of a ring. The portion of the second tube system 3 that routes the second feed stream to the second tube 4 can be configured in any manner consistent with the location and orientation of the second inlet 2, second tube 4, first tube 8 and first tube system 7, and can include multiple tubes, tube manifolds or combinations thereof.

First and second tubes 8, 4 can be arranged in separate horizontal planes or substantially in the same horizontal plane, and are concentric. For the purposes of this specification, the term “substantially in the same horizontal plane” means that the bottom surface of the tubes are separated by a vertical distance no greater than one outside tube diameter of the smaller of the tubes. Preferably, first and second tubes 8, 4 are arranged so that they lie substantially in the same horizontal plane.

Preferably, the concentric tubes are located adjacent to one another with minimum separation consistent with mechanical design considerations. The concentric tubes are positioned to provide uniform radial distribution of the feed streams, and are preferably positioned in the bottom portion of the reactor 1. More preferably, the concentric tubes are positioned in or proximate to reactor bottom head 26.

The first and second tubes 8, 4 can have the same or different diameters, depending on the relative flow rates of the respective feed streams, as would readily be recognized by one skilled in the art.

First tube 8 and second tube 4 include outlets 9, 5 for releasing the first and second fluids into the reactor 1. Outlets 9, 5 can include orifices, nozzles or combinations thereof. Outlets 9, 5 can be located anywhere on the outer surfaces of first and second tubes 8, 4, and are positioned so that, upon release of the first and second fluids into the reactor 1, they are directed toward each other, as shown in FIGS. 1A, 2, 9A and 10. Preferably, outlets 9, 5 are located on the bottom side of first and second tubes 8, 4 facing the reactor bottom head 26, to minimize fouling during reactor downtime or upset conditions. Another advantage of positioning outlets 9, 5, on the bottom side of first and second tubes 8, 4 is that the feed stream plume exiting outlets 9, 5 can minimize solids accumulation in the reactor bottom head 26. Preferably, outlets 9, 5 are proximate so that the plumes of the first and second fluids mix upon introduction into reactor 1.

Preferably, each outlet 9 on first tube 8 is positioned so that the exiting fluid is directed toward the nearest outlet 5 on second tube 4, and each outlet 5 on second tube 4 is positioned so that the exiting fluid is directed toward the nearest outlet 9 on first tube 8. This feature enhances initial mixing of the first and second feed streams after the fluids are in contact with the slurry, and promotes the formation of individual bubbles having the desired oxygen/hydrogen ratio at formation. Preferably, the angle of the outlet 9 with respect to the horizontal axis of first tube 8, and the angle of the outlet 5 with respect to the horizontal axis of second tube 4 ranges from 5 to 85°, more preferably 20 to 70°, most preferably, 30 to 60°. Lower angles risk a situation where the feed stream from one outlet would be directed into the other outlet, thereby creating the possibility of flammable mixtures. Lower angles also decrease the ability of the feed streams plume to prevent solids accumulation on the reactor bottom head 26. If the angle is too great, then a reduced amount of initial mixing occurs.

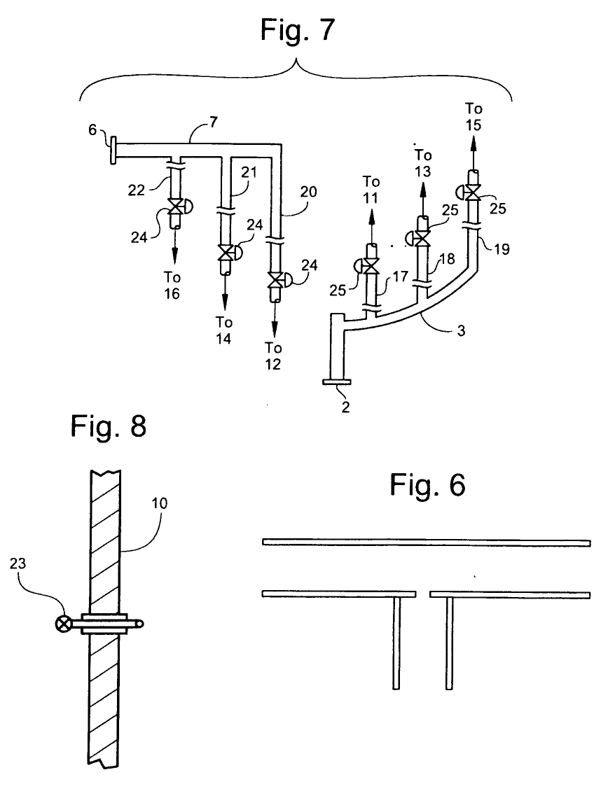

Outlets 9, 5 should have a minimum pressure drop to ensure proper fluid flow even during localized pressure fluctuations induced by the slurry circulation in reactor 1. Preferably, pressure drop across the outlets is from 5 to 20 psi. Fluid velocities exiting outlets 9, 5 are high enough to prevent solids accumulation in the reactor bottom head 26, but not high enough to cause excessive catalyst attrition or erosion in the reactor bottom head 26. Preferably, velocities are in the range of 50 to 120 ft/s, more preferably, 60 to 110 ft/s. Preferably, a shroud is installed on outlets 9, 5 to lower exit velocities, as shown in FIG. 6. When the outlets 9, 5 are orifices, preferably the orifice diameter is between 1 and 50 mm, more preferably, between 5 and 20 mm.

Preferably, the orifice density on first and second tubes 8, 4, defined as the number of orifices per m² of cross sectional area of reactor 1, is between 5 and 50, more preferably, between 10 and 25.

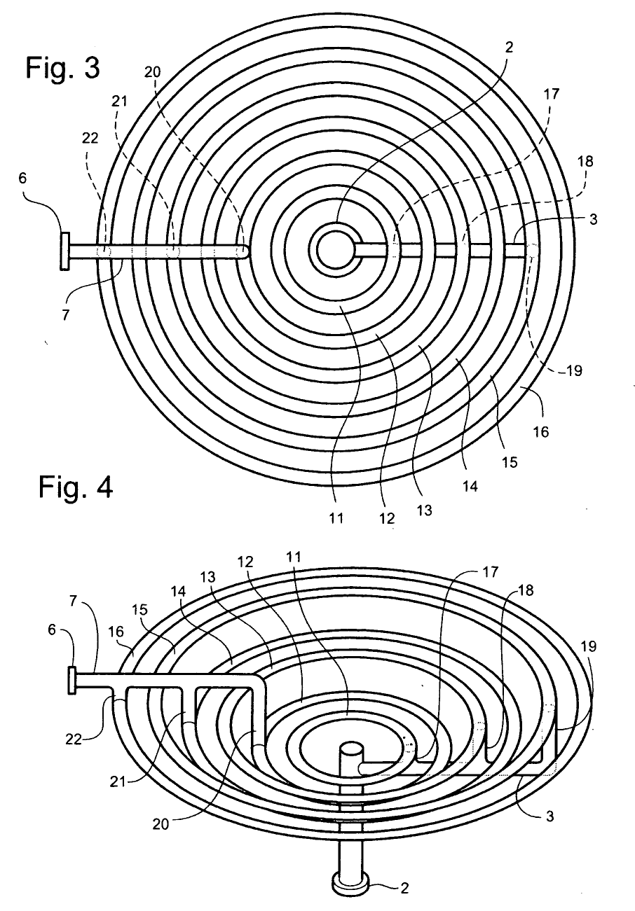

FIGS. 3 and 4 illustrate a preferred embodiment where the first and second tube systems 7, 3 contain multiple first and second tubes in the form of rings. First tube system 7 contains manifold tubing 20, 21 and 22, which routes the first feed stream to first tubes 16, 14, and 12. Second tube system 3 contains manifold tubing 17, 18, and 19 which routes the second feed stream to second tubes 11, 13, and 15. Each of first tubes 16, 14, and 12 are concentric with and preferably lie substantially in the same horizontal plane as second tubes 15, 13, and 11, thereby forming 3 ring pairs. Preferably, the 3 ring pairs are located in the bottom portion of the reactor 1, more preferably, in or proximate to the reactor bottom head 26. The multiple ring pairs can also be arranged so that there is varying vertical distances between them. The number and configuration of the outlets on the ring pairs are as described above.

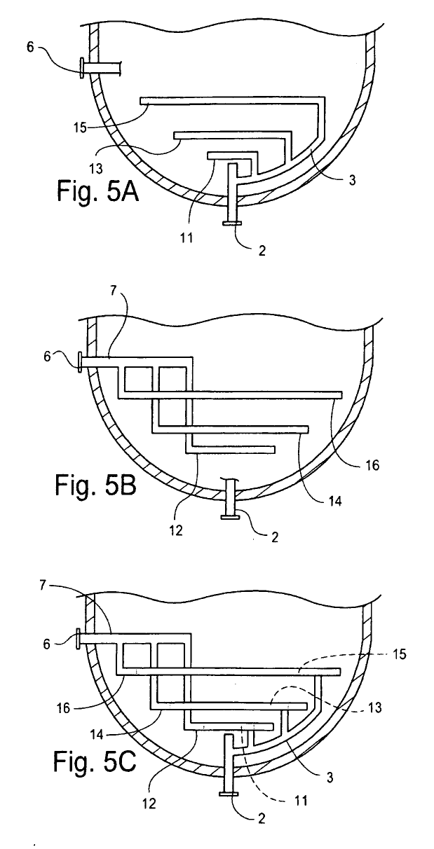

In a preferred embodiment, a multiple ring pair arrangement is located in or proximate to the reactor bottom head 26, with the innermost ring pair, 11, 12, being positioned lower than ring pair 13, 14, which is positioned lower than ring pair 15, 16. Because of the contour of the reactor bottom head 26, each of the ring pairs are substantially the same vertical distance from the reactor bottom head 26, as shown in FIGS. 5A-5C. For the purposes of this specification, the term “substantially the same vertical distance” means that the distance from the reactor bottom head 26 to the bottom surface of the ring pairs differs by no more than 2 outside diameters of the smallest tube of the ring pairs.

In another embodiment, the number of first and second tubes, and the distance between the ring pairs can be selected so that the ring pairs extend axially from the bottom portion of reactor 1 into the top portion of the reactor 1, to facilitate introduction of the feed streams throughout reactor 1. When ring pairs are located throughout reactor 1, control valves 24 can be present in the first tube system 7 and/or control valves 25 can be present in the second tube system 3 to allow selective control of the individual feed streams, as shown in FIG. 7. Such control valves 24, 25 can be modulated based on reactor conditions or the concentrations of reactant or product streams measured at various points in the reactor 1 by analyzer(s) 23, as shown in FIG. 8.

In another embodiment, the present subject matter relates to a process for producing propylene oxide in a reactor using the gas distribution system as described above, the process comprising feeding a first fluid into a reactor containing a solvent and a catalyst through a first tube system contained within the reactor, and a second fluid into the reactor through a second tube system contained within the reactor, where the first tube system and the second tube system are positioned so that, upon release into the reactor, the first and second fluids are directed towards each other. The first fluid is an oxygen-containing stream and the second fluid contains propylene, hydrogen, ballast gas, inerts and oxygen, as described above. Propylene oxide is produced in a reaction involving the first and second fluids, and is subsequently separated in downstream equipment.

The following example illustrates how the described gas distribution system would be expected to function.

TABLE 1

| | Second Tube | First Tube |
|---|---|---|
| Diameter of orifice, mm | 19.05 | 6.35 |
| Number of orifice | 501 | 539 |
| Orifice jet velocity, ft/s | 99 | 99 |
| ΔP across gas distributor, psi | 6 | 6 |
| Orifice density (number of orifices | 16 | 17 |
| per m² cross sectional area of the | | |
| SBCR) | | |

| | |
|---|---|
| Total gas flow rate to SBCR, scfs | 3261 |
| Superficial gas velocity, ft/s | ~0.5 |
| SBCR ID, ft | 21 |

We claim:

1. A device for reacting fluids comprising:
a reactor;
a first inlet for transporting a first fluid into the reactor;
a first tube system contained within the reactor and connected to and communicating with the first inlet for receiving the first fluid from the first inlet, the first tube system comprising at least one first tube in the form of a ring,
the at least one first tube comprising outlets for releasing the first fluid into the reactor;
a second inlet for transporting a second fluid into the reactor; and
a second tube system contained within the reactor and connected to and communicating with the second inlet for receiving the second fluid from the second inlet, the second tube system comprising at least one second tube in the form of a ring,
the at least one second tube comprising outlets for releasing the second fluid into the reactor;
wherein at least one of the first tubes and at least one of the second tubes are concentric, and the outlets for releasing the first fluid from the at least one first tube and the outlets for releasing the second fluid from the at least one second tube are positioned so that, upon release of the first and second fluids, the first and second fluids are directed towards each other.

2. The device of claim 1 wherein the at least one of the first tubes and the at least one of the second tubes lie substantially in the same horizontal plane.

3. The device of claim 1 wherein the reactor is a column having a top portion, a bottom portion, and a sidewall portion, and the at least one first tube and the at least one second tube are arranged at a position within the bottom portion of the reactor.

4. The device of claim 3 wherein
the at least one first tube and the at least one second tube each comprise a top side and a bottom side, the top side of the first and second tubes facing the top portion of the reactor, and the bottom side of the first and second tubes facing the bottom head of the reactor, and
the outlets for releasing the first fluid from the at least one first tube and the outlets for releasing the second fluid from the at least one second tube are each positioned on the bottom side.

5. The device of claim 4 wherein the first inlet for transporting the first fluid into the reactor is positioned at the sidewall portion of the reactor and the second inlet for transporting the second fluid into the reactor is positioned at the bottom portion of the reactor.

6. The device of claim 2 wherein the outlets for releasing the first fluid from the at least one first tube and the outlets for releasing the second fluid from the at least one second tube are positioned at angles of from 5 to 85 degrees relative to the horizontal plane of the respective first and second tubes.

7. The device of claim 1 wherein the outlets for releasing the first fluid from the at least one first tube and the outlets for releasing the second fluid from the at least one second tube further comprise a shroud.

8. The device of claim 1 wherein the first tube system further comprises a control valve.

9. The device of claim 1 wherein the second tube system further comprises a control valve.

10. A process comprising:
feeding a first fluid into a reactor containing a solvent and a catalyst, through a first tube system contained within the reactor, the first fluid comprising oxygen; and
feeding a second fluid into the reactor through a second tube system contained within the reactor, thereby forming propylene oxide, the second fluid comprising propylene and hydrogen,
wherein the first tube system and second tube system are positioned such that, upon release into the reactor, the first and second fluids are directed towards each other.
